(19) United States
(12) Patent Application Publication
(10) Pub. No.: US 2002/0155087 A1
(43) Pub. Date: Oct. 24, 2002

(54) Instant air freshener composition

(76) Inventors: Lyn Ryan, Reynoldsburg, OH (US); Maxine Pesu, Gahanna, OH (US)

Correspondence Address: NOTARO AND MICHALOS, 100 DUTCH HILL ROAD, SUITE 110, ORANGEBURG, NY, 10962-2100, US

(21) Appl. No.: 09/793,866

(22) Filed: Feb. 27, 2001

(51) Int. Cl. A61L 9/01

(52) U.S. Cl. 424/76.4

(57) ABSTRACT

An air freshening composition is provided in non-aqueous form for mixing with water prior to use. The composition is composed of a super-absorbent polymer, a granular preservative, fragrance and, optionally, a dye. The composition is packaged and stored in sealed containers until opened for use. The composition is particularly well suited for reusing empty slow diffuser type air freshening containers.

### FIELD AND BACKGROUND OF THE INVENTION

The present invention relates generally to the field of slow diffusers and in particular to a new and useful non-aqueous composition which can be mixed with water in a container to produce a slow diffusing air freshener.

Slow diffusing air fresheners are generally known. Typically, an air freshening composition is provided in a container to give shape to the composition and hold it together. The composition is exposed to the air and allowed to gradually evaporate, thereby releasing a fragrant scent to the air.

U.S. Pat. No. 5,556,835 discloses an aqueous gel fragrance composition. The composition includes an aqueous fluid, a gelling agent and an oil-absorbable resin carrying a fragrant material. The oil-absorbable resin must be one capable of absorbing the fragrant material, such as hydrophobic cross-linked polymers containing unsaturated monomers.

U.S. Pat. No. 5,145,673 discloses a gel liquid deodorizing composition for cigarette butts having water, a water-absorbing polymer and a viscosity-imparting agent. The viscosity-imparting agent can be any compound which increases the viscosity of the composition, but glycols are preferred.

A granular solid deodorant composition having a hydrated calcium sulfate base impregnated with fragrance oils and microcapsules containing additional fragrance oils is taught by U.S. Pat. No. 5,164,178. The base and microcapsules are contained in a sealed pouch having a porous side which can be uncovered to release the deodorant fragrance.

U.S. Pat. No. 4,209,417 teaches perfumed particles formed from 30-70% of water-insoluble perfume, 25-65% water-soluble polymer and an emulsifier. The particles are a polymeric matrix with uniform droplets of perfume dispersed throughout the matrix. The emulsifier makes the droplets uniform. The composition is particularly useful in laundry detergents.

A solid air freshener consisting of a granular foam phase dispersed in a gelled phase is disclosed in U.S. Pat. No. 5,034,222. The foam phase includes hydrophilic polyurethane having a volatile air freshening substance, such as fragrance oils, a surfactant which is soluble or dispersable in water, and a solid filler material.

U.S. Pat. No. 5,679,334 discloses a gel composition formulation of water-based air freshener used in JELLY JARS air fresheners including a botanical element sold by Bath & Body Works, Inc. The gel composition includes a cross-linking agent and co-solvent for the fragrance oil.

### SUMMARY OF THE INVENTION

It is an object of the present invention to provide an air freshener composition which is non-aqueous during manufacture and distribution and can be activated by mixing with water.

It is a further object of the invention to provide an air freshener composition which can be used by consumers to quickly and easily replenish empty air freshener devices.

Accordingly, an instant air freshener of the invention comprises a water-based air freshener which is provided in a chunky, or granular, form in foil or other type of sealed package or container. The air freshener is activated by adding water to the granular form.

The granular form of the air freshener is composed of 50-90% of a super-absorbent polymer, 3-10% of a granular preservative, 10-50% fragrance oils and FD&C powder dye for coloring as needed. The granular air freshener can be activated by combining it with water in a ratio of about 7 grams of air freshener to one cup of water.

A notable difference with other volatile air fresheners is that the fragrance oil and water can be combined without need for a solubulizer.

The various features of novelty which characterize the invention are pointed out with particularity in the claims annexed to and forming a part of this disclosure. For a better understanding of the invention, its operating advantages and specific objects attained by its uses, reference is made to the accompanying descriptive matter in which a preferred embodiment of the invention is illustrated.

### DESCRIPTION OF THE PREFERRED EMBODIMENTS

The composition of the invention is a non-aqueous granular homogenous mixture which can be combined with water to provide a good slow diffusing air freshening gel. The composition and water are mixed in a container to make and hold the air freshener gel for diffusing. In a preferred embodiment, the composition is used to replenish empty slow diffuser air freshener containers, thereby recycling the container for further use.

The non-aqueous composition contains the ingredients below in the amounts shown in the following table:

| Ingredient | Range | Preferred |
|---|---|---|
| Granular Super- | 50-87% wt. | 50-60% wt. |
| Absorbent Polymer | | |
| Granular | 3-10% wt. | 5-10% wt. |
| Preservative | | |
| Liquid Dye | q.s. | 1-2% wt. |
| Fragrance Oil | 10-47% wt. | 34-44% wt. |

To make the non-aqueous composition, the liquid dye is mixed into the fragrance oil with the granular preservative. Once a homogenous mixture of fragrance oil, dye and preservative has been obtained, it is combined with the granular super-absorbent polymer and mixed thoroughly. The liquid dye and fragrance oil wet the two granular powder ingredients so that the ingredients will form a gelatinous, chunky mixture. The chunky mixture resembles a plurality of irregularly shaped crystals of JELLO. The chunky mixture is not a continuous liquid or solid, but rather, is composed of many irregular pieces.

The chunky mixture can be stored in a sealed container for later use. Suitable containers include, bottles and jars with lids, foil packages with heat sealed edges, and resealable plastic bags. Resealable containers are preferred for storing the mixture so that only a desired amount need be used at any one time and the remainder can be returned to storage. The containers should be airtight to prevent moisture from entering the container and activating the chunky mixture to form a gel air freshener.

Alternatively, water can be added to the chunky mixture immediately to form a gel air freshener.

Whether the chunky mixture is stored for some time first or used immediately, when it is desired to activate the chunky mixture and make the gel air freshener, the chunky mixture is simply combined with water. A preferred range for the ratio of chunky mixture to water is between about 3-11 grams of chunky composition to 1 cup (8 fl. oz.) of water, and a most preferred ratio is about 7 grams of the chunky composition to 1 cup (8 fl. oz.) of water. The water and mixture are combined in any suitably sized container. The container used should be the one the gel air freshener will be held in for use. After about 15 minutes, the gel air freshener will be set and ready for use.

The granular super-absorbent polymer is preferably one such as AP 41K made by Stockhausen, Inc. of Greensboro, N.C. This particular super-absorbent polymer is a crosslinked potassium polyacrylate/polyacrylamide copolymer provided as particles sized from about 1000-3000 microns. The copolymer is a white powder when dry. The super-absorbent polymer swells in the presence of water and other liquids to form a gel-like suspension.

Other acceptable super-absorbent polymers include WATERLOCK from Grain Processing Corporation, also known by its INCI name, corn starch/acrylamide/sodium acrylate copolymer.

A preferred preservative is one such as DANTOGARD PLUS manufactured by Lonza of Fairlawn, N.J. DANTOGARD PLUS is a mixture of hydantoin derivative compounds in the form of a white crystalline powder.

The instant air freshener composition can be used, for example, to replenish the air freshener used in Bath & Body Works' JELLY JARS air freshener products. In one formulation of the instant air freshener used for this purpose, 53% polymer is combined with 7% preservative, 1% colorant and 39% fragrance oil to produce a granular air freshener mix. In a preferred embodiment, the air freshener mix can be combined with water in a ratio of 7 g mix per cup (8 fl. oz.) of tap water to activate the air freshener. It should be noted that it is acceptable to combine the mix with one cup of water in ratios between 3 g to 11 g mix per cup. Using less mix will result in a watery product, while more mix provides a more solid product.

The instant air freshener composition is different from other known compositions in that the fragrance oil is premixed with the other components, and so a solubilizer is not required to dissolve the fragrance oil in the water added to change the chunky mixture to a gel. This difference permits the elimination of an additional chemical which can affect the appearance and other properties of the air freshener.

While a specific embodiment of the invention has been shown and described in detail to illustrate the application of the principles of the invention, it will be understood that the invention may be embodied otherwise without departing from such principles.

What is claimed is:

1. An instant air freshener composition comprising:
a mixture of about 50-87% by weight of a granular super-absorbent polymer, about 3-10% by weight of a granular preservative, about 10-47% by weight of a fragrance oil, and a liquid dye, the mixture being provided as a plurality of individual chunks.

2. An instant air freshener composition according to claim 1, wherein the mixture is about 50-60% by weight of super-absorbent polymer, about 5-10% by weight of a granular preservative, about 34-44% by weight of a fragrance oil, and 1-2% by weight of a liquid dye.

3. An instant air freshener composition according to claim 2, wherein the super-absorbent polymer is a crosslinked potassium polyacrylate/polyacrylamide copolymer.

4. An instant air freshener composition according to claim 3, wherein the preservative comprises a mixture of hydantoin derivative compounds.

5. An instant air freshener composition according to claim 3, wherein the preservative comprises a mixture of hydantoin derivative compounds.

6. An instant air freshener for activating with water prior to use, the air freshener comprising:
a sealable container; and
a mixture of about 50-87% by weight of a granular super-absorbent polymer, about 3-10% by weight of a granular preservative, about 10-47% by weight of a fragrance oil, and a liquid dye, the mixture being provided as a plurality of individual chunks in the container.

7. An instant air freshener according to claim 6, wherein the mixture is about 50-60% by weight of super-absorbent polymer, about 5-10% by weight of a granular preservative, about 34-44% by weight of a fragrance oil, and 1-2% by weight of a liquid dye.

8. An instant air freshener according to claim 7, wherein the super-absorbent polymer is a crosslinked potassium polyacrylate/polyacrylamide copolymer.

9. An instant air freshener according to claim 8, wherein the preservative comprises a mixture of hydantoin derivative compounds.

10. An instant air freshener according to claim 7, wherein the preservative comprises a mixture of hydantoin derivative compounds.
